(19) United States
(12) Patent Application Publication
(10) Pub. No.: US 2019/0142555 A1
(43) Pub. Date: May 16, 2019

(54) SYSTEM FOR ACQUISITION OF ORAL CAVITY ULTRASOUND IMAGES

(71) Applicant: Sonodontics, Wilmington, DE (US)

(72) Inventors: Jonathan Macoskey, Ann Arbor, MI (US); Jonathan Lundt, Ann Arbor, MI (US)

(21) Appl. No.: 16/185,329

(22) Filed: Nov. 9, 2018

Related U.S. Application Data
(60) Provisional application No. 62/584,315, filed on Nov. 10, 2017.

(51) Int. Cl. A61C 9/00; A61B 8/00; A61B 8/12; A61B 8/08

(52) U.S. Cl. CPC A61C 9/0086; A61B 8/54; A61B 8/12; A61B 8/4411; A61B 1/24; A61B 8/483; A61B 8/4281; A61B 8/56; A61B 8/0875; A61B 8/4461

(57) ABSTRACT

Various systems and methods for imaging soft oral tissue via an ultrasound oral imaging instrument are disclosed. The ultrasound oral imaging instrument can be coupled to an ultrasound probe that includes an ultrasonic transducer for generating ultrasound pulses. The instrument can include an actuator couplable to the ultrasound probe. The actuator can move the ultrasound probe as the as the ultrasound probe emits ultrasound pulses to image tissue against which the ultrasound probe is directed. The instrument can be utilized to image soft oral tissue by applying an acoustic coupler to the soft oral tissue to be imaged or the ultrasound probe, moving the ultrasound probe along the soft oral tissue to captured two-dimensional slices or images of the tissue, and then reconstructing three-dimensional images from the two-dimensional slices.

### CROSS-REFERENCE TO RELATED APPLICATIONS

The present application claims priority under 35 U.S.C. § 119(e) to U.S. Provisional Patent Application No. 62/584,315, titled SYSTEM FOR ACQUISITION OF ORAL CAVITY ULTRASOUND IMAGES, filed on Nov. 10, 2017, the disclosure of which is herein incorporated by reference in its entirety.

### BACKGROUND

Medical imaging has shown rapid technological advancement in image resolution and quality, ease-of-use, and cost effectiveness over the past decade. In the dental industry, these advancements have led to reductions in oral diseases, such as tooth decay and tooth misalignment. Currently, the main imaging modalities in dentistry are two-dimensional (2D) plane x-ray and x-ray computed tomography (CT). While these modalities are well-suited for hard tissue (e.g., bone and teeth), they have limited utility for soft tissue (e.g., gingiva). Characterization of soft tissue is crucial given that gum inflammation (gingivitis) and gum disease (periodontitis) are highly prevalent, affecting half of adults over the age of 30 years in the U.S. Numerous studies have shown that poor gum health can lead to early tooth loss and decay and is associated with a variety of systemic health issues including heart disease, diabetes, and stroke. Because the disease is limited to the soft-tissue structures, it is often not clinically diagnosable via x-ray images. The only way to evaluate these structures is by probing the gum line using a metal probe to check for the periodontal pocket depth, as is done at a routine dental visit. However, this technique is very painful for patients, has poor sensitivity and specificity, and is susceptible to high inter-practitioner variability.

Medical ultrasound imaging is an imaging technique that uses acoustic waves generally on the order of megahertz (MHz) in frequency to image structures in the body. In contrast to x-rays, ultrasound waves are attenuated and reflected differentially by subtle heterogeneities in soft tissues. This makes ultrasound ideal for soft tissue imaging. Today, ultrasound continues to be a major imaging modality used in the medical field to examine many organs including the heart, liver, kidneys, and male and female reproductive organs. However, dental practitioners have yet to adopt oral cavity ultrasound despite its high potential. This is due in part to limitations in technology to develop an ultrasound probe small enough to be used in the oral cavity but also the need for supporting hardware and software to facilitate the acquisition of data that are easy for the practitioner to interpret and utilize. Described herein are systems and methods employing ultrasound imaging techniques in order to image soft tissue in the oral cavity for tracking the progression of diseases, such as periodontitis, and other applications. A high-frequency ultrasound transducer integrated with an actuation device allows for consistent image acquisition, which may result in high-resolution, three-dimensional (3D) ultrasound images of oral soft tissue of the oral cavity.

### SUMMARY

In one general aspect, an ultrasound oral imaging instrument is disclosed. The ultrasound oral imaging instrument includes an actuator couplable to an ultrasound probe and a control circuit. The actuator is configured to move the ultrasound probe between a first position and a second position. The control circuit is couplable to the ultrasonic transducer of the ultrasound probe and coupled to the actuator. The control circuit is configured to cause the ultrasonic transducer to emit ultrasound pulses through the ultrasound probe and cause the actuator to move the ultrasound probe between the first position and the second position as the ultrasound probe emits the ultrasound pulses.

In another general aspect, a method for obtaining oral images via an ultrasound oral imaging instrument is disclosed. The method includes applying an acoustic coupler between a gingival tissue of a patient and an ultrasound probe of the ultrasound oral imaging instrument; moving the ultrasound probe along the gingival tissue, the ultrasound probe comprising a ultrasonic transducer configured to emit an ultrasonic pulse; capturing a plurality of 2D images via the ultrasound probe as the ultrasound probe is moved along the gingival tissue; and reconstructing the plurality of 2D images into a 3D image of the gingival tissue.

### FIGURES

The features of various aspects are set forth with particularity in the appended claims. The various aspects, however, both as to organization and methods of operation, together with further objects and advantages thereof, may best be understood by reference to the following description, taken in conjunction with the accompanying drawings as follows.

FIG. 1 is a sectional view of an ultrasound oral imaging instrument, in accordance with at least one aspect of the present disclosure.

FIG. 2 is an underside plan view of the ultrasound oral imaging instrument illustrated in FIG. 1, in accordance with at least one aspect of the present disclosure.

FIG. 3 is a side plan view of the ultrasound oral imaging instrument illustrated in FIG. 1, in accordance with at least one aspect of the present disclosure.

FIG. 4 is a sectional view of a probe of the ultrasound oral imaging instrument, in accordance with at least one aspect of the present disclosure.

FIG. 5 is a block diagram of an ultrasonic oral imaging system, in accordance with at least one aspect of the present disclosure.

FIG. 6 is a sectional view of an ultrasound oral imaging instrument indicating a center of gravity of the instrument, in accordance with at least one aspect of the present disclosure.

FIG. 7 is a view of a generator for powering the ultrasound oral imaging instrument, in accordance with at least one aspect of the present disclosure.

FIG. 8 is a perspective view of the ultrasound oral imaging instrument connected to the generator, in accordance with at least one aspect of the present disclosure.

FIG. 9 is a plan view of the ultrasound oral imaging instrument imaging along a gingival surface, in accordance with at least one aspect of the present disclosure.

FIG. 10 is a sectional view along line 10-10 of the ultrasound oral imaging instrument illustrated in FIG. 9, in accordance with at least one aspect of the present disclosure.

FIG. 11 is a view of an ultrasound oral imaging instrument that includes a stabilizer, in accordance with at least one aspect of the present disclosure.

FIG. 12 is a view of the ultrasound oral imaging instrument of FIG. 11 imaging along a gingival surface, in accordance with at least one aspect of the present disclosure.

FIG. 13 is a view an ultrasound oral imaging instrument with a general configuration similar to a mouth piece, in accordance with at least one aspect of the present disclosure.

FIG. 14 is a sectional view along line 14-14 of the ultrasound oral imaging instrument illustrated in FIG. 13, in accordance with at least one aspect of the present disclosure.

### DESCRIPTION

Certain aspects will now be described to provide an overall understanding of the principles of the structure, function, manufacture, and use of the devices and methods disclosed herein. One or more examples of these aspects are illustrated in the accompanying drawings. Those of ordinary skill in the art will understand that the devices and methods specifically described herein and illustrated in the accompanying drawings are non-limiting examples aspects and that the scope of the various aspects is defined solely by the claims. The features illustrated or described in connection with one aspect may be combined with the features of other aspects. Such modifications and variations are intended to be included within the scope of the claims. Furthermore, unless otherwise indicated, the terms and expressions employed herein have been chosen for the purpose of describing the illustrative aspects for the convenience of the reader and are not to limit the scope thereof.

In many dental imaging applications, x-ray imaging or other x-ray-based imaging modalities are generally used. These imaging modalities are ideal for imaging hard structures in the mouth such as teeth or the maxillary bone. However, they are poorly suited for imaging soft tissue. One advantage of the systems and methods utilizing an ultrasound oral imaging instrument that are described herein is that ultrasound imaging has the capability of producing very high-resolution images of soft tissue in the oral cavity, due in part to the short acoustic signal path. A short acoustic signal path reduces the effects of acoustic attenuation, which in turn allows high frequency (i.e., >10 MHz, generally) sound waves to be utilized. Accordingly, ultrasound imaging is highly advantageous for imaging soft oral tissue.

Further, 3D ultrasound imaging applications, in general, require an ultrasound imaging probe that is built to acquire 3D images without moving the probe. However, these 3D imaging probes are generally relatively expensive and also generally require a relatively large amount of operating space. Another advantage of the systems and methods utilizing an ultrasound oral imaging instrument that are described herein is that 3D images can be reconstructed from 2D ultrasound images acquired via an ultrasound imaging instrument. By reconstructing 3D images from 2D images, the presently described system obviates the need for specialized 3D imaging probes to acquire 3D images.

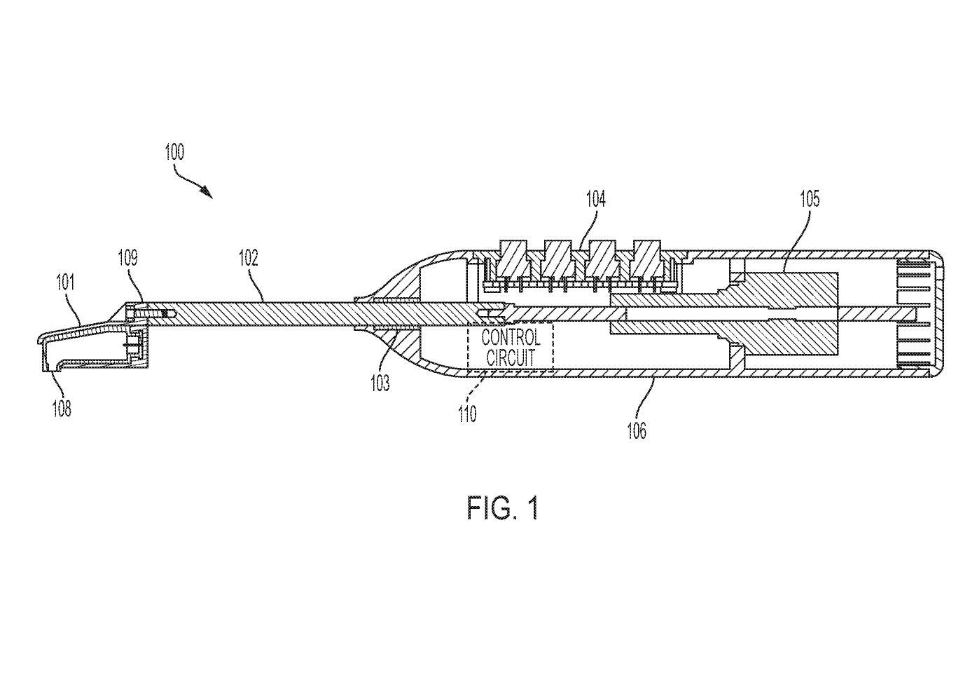

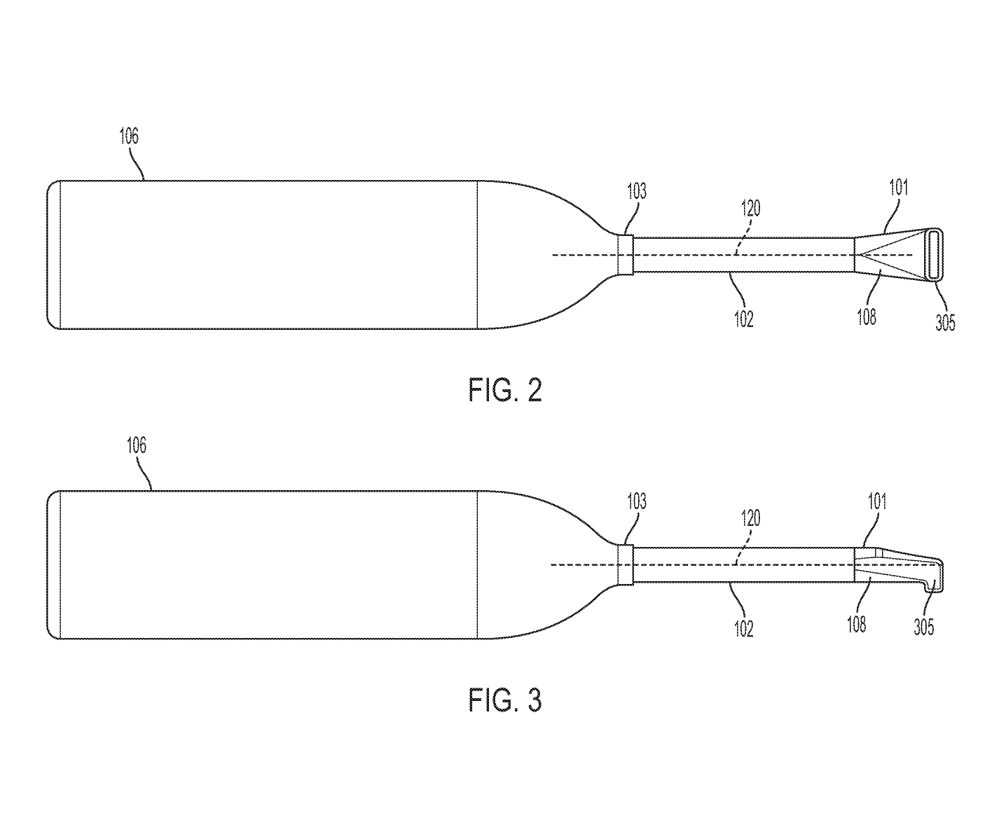

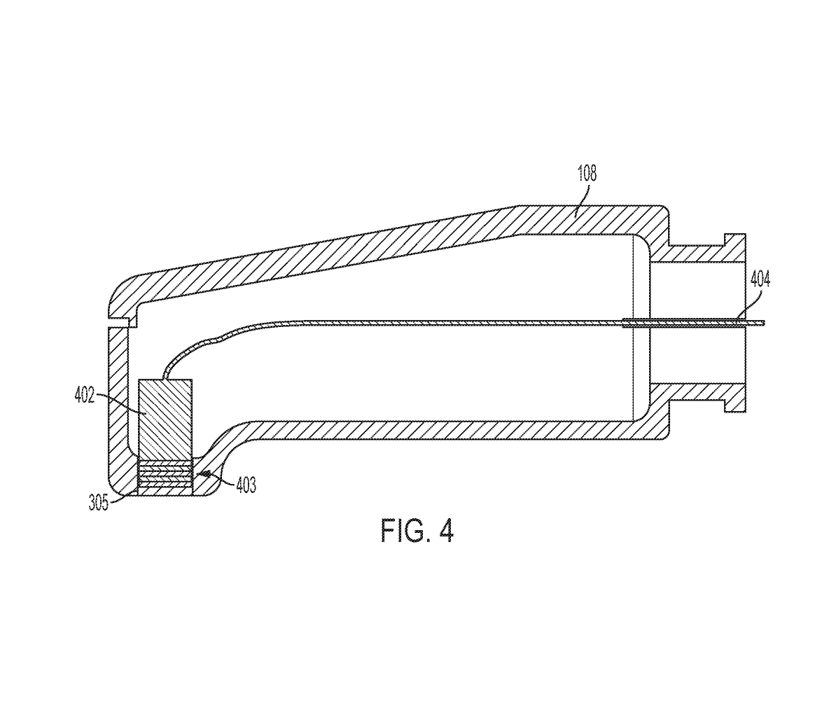

FIGS. 1-4 illustrate various views of an ultrasound oral imaging instrument 100, in accordance with at least one aspect of the present disclosure. The ultrasound oral imaging instrument 100 includes a housing 106 enclosing an actuator 105 (e.g., a linear actuator) and a shaft 102 that extends through an opening 103 in the housing 106 and is coupled to the actuator 105. In various aspects, the housing 106 may cylindrical in shape or a variety of other shapes depending upon the desired ergonomics of the ultrasound oral imaging instrument 100. In the depicted aspect, the shaft 102 has a linear, rod-like shape; however, in other aspects the shaft 102 may have other, nonlinear shapes. The shaft 102 can be constructed from a variety of materials, such as varieties of steel, stainless steel, aluminum, titanium, and plastics such as acetal, polyether ether ketone (PEEK), polycarbonate, or other polymers. Metal components may or may not have a surface coating, such as anodization or electroplating. In one aspect depicted in FIGS. 1-4,6, and 8, the distal end 109 of the shaft 102 includes a mount 101 that is configured to hold or kinematically couple an ultrasound probe 108 to the ultrasound oral imaging instrument 100. The mount 101 can include a clip, for example. In one aspect, the mount 101 can be removably connected to the shaft 102. For example, the mount 101 can be attached to the shaft via a screw that is configured to engage with corresponding threading disposed within the shaft 102. Having the ultrasound probe 108 and/or mount 101 be removably attachable allows those components to be replaced and/or sterilized after each use of the ultrasound oral imaging instrument 100, for example. In another aspect, the mount 101 can be fixedly or integrally affixed to the shaft 102. In another aspect, the ultrasound probe 108 can be fixedly or integrally affixed to the distal end 109 of the shaft 102.

The ultrasound probe 108 can include an ultrasonic transducer 402 that is configured to produce ultrasound waves or pulses that propagate through and are reflected by the tissue against which the ultrasound probe 108 is situated. The ultrasound probe 108 then receives the reflected sound waves. Images of tissues or structures underlying the surface of the tissue can be constructed by evaluating the reflected sound signals, which can correspond to the different acoustic impedances of the underlying materials. For example, the length of time between when a sound signal is initially transmitted and when the corresponding reflected sound signal is received (i.e., the time-of-flight of the sound signal) can be utilized to determine the depth of a structure from which the sound signal was reflected. In one aspect, the ultrasound probe 108 is driven at a frequency between 10-65 MHz. In another aspect, the ultrasound probe 108 is driven at a frequency of at least 20 MHz. In some aspects, the ultrasound probe 108 can include an ultrasonic transducer 402 comprising one or more piezoelectric elements 403 that vibrate according to a drive signal provided by an electrical connection 404. The piezoelectric elements 403 can be arranged in a number of different configurations, including being arranged in stacks or phased arrays (e.g., linear, rectangular, annular, or circular arrays of piezoelectric elements 403). In some aspects, the piezoelectric elements 403 in the ultrasonic transducer 402 may vibrate in- or out-of-phase with one another. In aspects where the piezoelectric elements 403 are arranged in a stacked configuration, the phase differences between the piezoelectric elements 403 can change the amplitude of the ultrasound pulses generated by the ultrasonic transducer 402, for example. In aspects where the piezoelectric elements 403 are arranged in phased arrays, the phase differences between the piezoelectric elements 403 can cause the ultrasound pulses generated by the ultrasonic transducer 402 to be emitted at different times and/or in different directions. In some aspects, the drive signal is provided via an onboard control circuit 110, whereas in other aspects the drive signal can be provided by an external generator to which the ultrasound oral imaging instrument 100 is connectable. As the piezoelectric elements 403 vibrate, the ultrasonic transducer 402 can transmit pulsed sound waves through an opening 305 in the ultrasound probe 108 to any tissue that it situated thereagainst. In one aspect, the ultrasound probe 108 can further include an acoustic lens and/or an acoustic matching layer (e.g., a quarter wavelength acoustic matching layer) situated over the opening 305. An acoustic matching layer and/or an acoustic lens can be beneficial in order to improve transmission of ultrasound pulses to any tissue that is situated thereagainst.

In various aspects, the actuator 105 can be configured to impart linear and/or rotational movement on the ultrasound probe 108 through the shaft 102 coupled to the actuator 105. In some aspects, the ultrasound oral imaging instrument 100 can include multiple actuators 105 for actuating the ultrasound probe 108 by and/or about multiple axes. In the depicted aspect, the actuator 105 is a linear actuator that is configured to actuate the ultrasound probe 108, the shaft 102, and/or the mount 101 along the longitudinal axis 120 of the shaft 102 between a first or proximal position and a second or distal position. The movement imparted by the actuator 105 can include unidirectional or reciprocating (i.e., oscillating) movement. The first position, the second position, any positions in between the first and second positions, and/or the rate at which the actuator 105 moves the ultrasound probe 108 or the shaft 102 between two positions can be predetermined, set by a control circuit 110 coupled to the actuator 105, or set by a user via user controls 104. In various aspects, the actuator(s) 105 can be configured to impart stepped or continuous motion. For example, the actuator(s) 105 can include a stepper motor, servo motor, piezo motor, pneumatic actuator, and so on. In one aspect, the actuator 105 is configured to impart motion that is precise to a sub-millimeter scale (e.g., the actuator 105 can include a step motor having sub-millimeter step sizes). The actuator 105 can be configured to impart a range of motion (i.e., the distance between the first position and the second position) on the ultrasound probe 108, the shaft 102, and/or the mount 101 of several centimeters or more, for example. The actuator 105 can also be configured to drive the ultrasound probe 108, the shaft 102, and/or the mount 101 at a rate of one cm/s or more, for example.

The ultrasound probe 108 can be configured to emit the sound pulses in different orientations or at a range of angles relative to the direction in which the ultrasound probe 108 is actuated. The angle at which the sound pulses are emitted by the ultrasound probe 108 can be controlled according to the orientation of the ultrasonic transducer 402 within the ultrasonic probe 108, utilizing phased arrays of piezoelectric elements 403, and so on. For example, in the aspect depicted in FIGS. 1-3, the opening 305 of the ultrasound probe 108 through which the sound pulses are transmitted is oriented perpendicularly to the direction of movement of the ultrasound probe 108 (i.e., the longitudinal axis 120 of the shaft 102). In other aspects, the ultrasound probe 108 can be configured to emit the sound pulses parallel to its direction of movement or in other orientations.

The ultrasound oral imaging instrument 100 further includes a control circuit 110 that is coupled to the actuator 105 and/or the ultrasound probe 108. The control circuit 110 can include combinations of hardware, firmware, and/or software components for performing the recited functions. Further, the ultrasound oral imaging instrument 100 can include a power source coupled to the control circuit 110 for powering and/or providing signals for driving various components of the ultrasound oral imaging instrument 100. In one aspect, the control circuit 110 is configured to provide a drive signal (e.g., an AC electrical signal) to the ultrasonic transducer 402 to cause the ultrasonic transducer 402 to emit ultrasound pulses. In one aspect, the control circuit 110 is coupled to the actuator 105 and is configured to control the actuator 105 in order to cause the actuator 105 to move the ultrasound probe 108 at a known rate as the ultrasound probe 108 emits the ultrasound pulses. As the ultrasound probe 108 moves, it sends and receives ultrasound pulses that can be utilized to construct 2D images of the tissue. Each of these images is a 2D “slice” of the tissue being visualized. Further, because the ultrasound probe 108 is moving between known positions at a known rate, the ultrasound probe 108 is thus taking multiple 2D slices of the underlying tissue at different positions (which are known because the ultrasound probe 108 is moving between known positions at a known rate). Accordingly, the 2D slices can be arranged according to their positions, and then 3D images of the underlying tissue can be constructed from the 2D slices, as is described in more detail below.

In one aspect, the ultrasound oral imaging instrument 100 can include controls 104 for controlling various functions or modes of the ultrasound oral imaging instrument 100. For example, the controls 104 can be coupled to the control circuit 110 and cause the ultrasound oral imaging instrument 100 to turn on and off, determine the direction and/or speed of movement of the ultrasound probe 108, determine when ultrasound data are being acquired, and so on. For example, the controls 104 can include buttons that cause the ultrasound probe 108 to move forward quickly, forward slowly, reverse slowly, and reverse quickly. In other aspects, the ultrasound oral imaging instrument 100 does not include any controls 104 and instead the control circuit 110 controls the functions of the ultrasound oral imaging instrument 100 according to predetermined or preprogrammed instructions. In some aspects, the control circuit 110 can be configured to control the ultrasound oral imaging instrument 100 according to instructions that are transmitted to, loaded onto, or otherwise provided to the ultrasound oral imaging instrument 100 from an external computer or electronic device.

In various aspects, the ultrasound oral imaging instrument 100 can be configured to receive power and/or instructions from an external source, such as a generator, a computer, or an electronic device, as described above. In such aspects, the ultrasound oral imaging instrument 100 can include various connectors for receiving power and/or data from the external source. Such connectors can include, for example, various wired connectors, such as USB Type-C connector or a power cord, or wireless connectors, such as a Bluetooth transceiver.

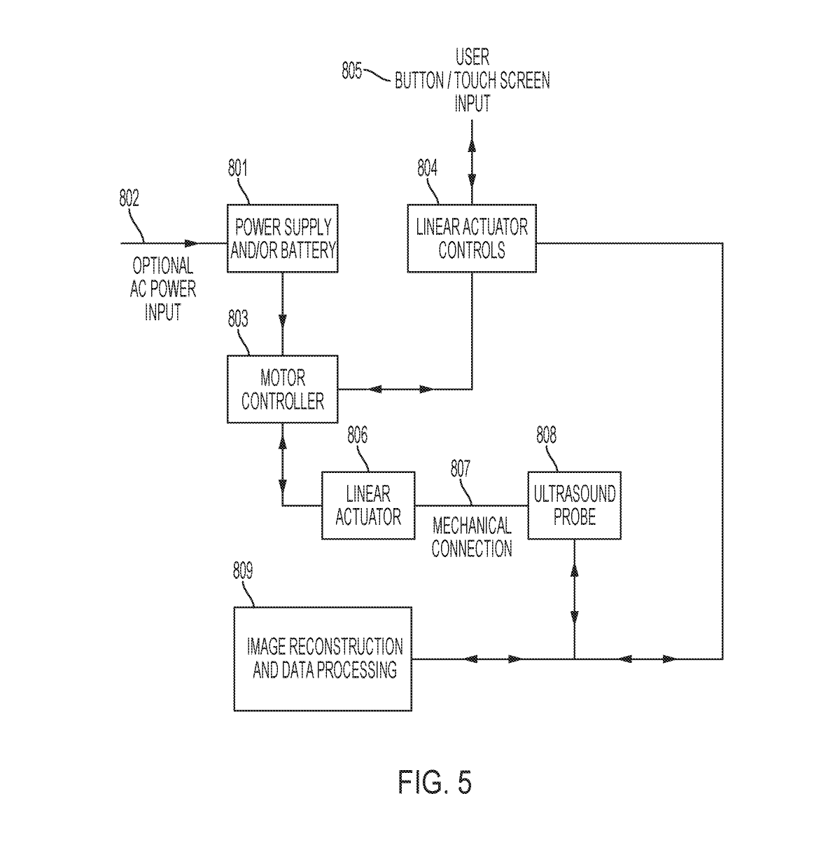

FIG. 5 is a block diagram of an ultrasonic oral imaging system, in accordance with at least one aspect of the present disclosure. The control circuit 110 described in connection with FIGS. 1-4 can include, for example, a motor controller 803 that is coupled to a linear actuator 806. The motor controller 803 can be configured to output motor data, which can in turn be utilized to measure and/or record the position of the ultrasound probe 808, shaft 102, and/or mount 101 that is being moved by the linear actuator 806. Further, the motor controller 803 can be coupled to a power supply 801 (e.g., a battery). In one aspect, the power supply 801 can be integral to the ultrasound oral imaging instrument 100 and optionally receive external power 802. In another aspect, the power supply 801 can include a generator external to the housing of the ultrasound oral imaging instrument 100, and, accordingly, the ultrasound oral imaging instrument 100 can be plugged into or otherwise connected to the generator to function. The motor controller 804 can be programmed by or receive instructions from linear actuator controls 804, which can in turn be controlled via user input received via user controls 805 (e.g., buttons or a touch screen), voice commands (detected by a microphone of the ultrasound oral imaging instrument 100 or external control device 502, for example), and other input methods. The linear actuator 806 is mechanically connected 807 to the ultrasound probe 808 (via, e.g., a shaft 102, as depicted in FIGS. 1-4) such that the linear actuator 806 can move the ultrasound probe 808 as the ultrasound probe 808 sends and receives ultrasound pulses.

The ultrasonic oral imaging system further includes an image reconstruction module 809 that is communicably coupled to the ultrasound probe 808 (e.g., via the electrical connection 404). In one aspect, the image reconstruction module 809 can receive the ultrasound signal data from the ultrasound probe 808, construct 2D images from the signal data, determine the position corresponding to each of the 2D images, and then construct 3D images from the 2D images utilizing a variety of 3D reconstruction algorithms. In one aspect, the image reconstruction module 809 can include, for example, an image processing program, which may be a custom image processing program written in a variety of computer languages, such as Python, C/C++, C#, Java, or JavaScript, for example. The image reconstruction module 809 can also or alternatively include a variety of particular algorithms, such as a volume rendering algorithm or a machine learning or deep learning algorithm. In some aspects, the image reconstruction module 809 is executed by a control circuit 110 of the ultrasound oral imaging instrument 100. In other aspects, the image reconstruction module 809 is executed by an external computer or electronic device to which the ultrasound oral imaging instrument 100 is communicably connectable. For example, the ultrasound oral imaging instrument 100 can store the ultrasound signal data in an onboard memory, which can then be downloaded to an external computer executing the image reconstruction module 809 from the ultrasound oral imaging instrument 100. As another example, the ultrasound oral imaging instrument 100 can transmit the captured ultrasound signal data to an external computer executing the image reconstruction module 809 via a wired or wireless connection. In some aspects, feedback from the image reconstruction module 809 can control various functions of the ultrasound probe 808 and/or the linear actuator controls 804. For example, if the image reconstruction module 809 determines that there was an error with obtaining a particular image or set of images, feedback from the image reconstruction module 809 can cause the ultrasound probe 808 to reactive, alter the frequency at which the ultrasound pulses are emitted, alter the orientation at which the ultrasound pulses are emitted, and so on. As another example, if the image reconstruction module 809 determines that there was an error with obtaining a particular image or set of images, feedback from the image reconstruction module 809 can cause the linear actuator controls 804 to move the ultrasound probe 108 back over the target area to be imaged, change a speed at which the linear actuator 806 drives the ultrasound probe 808, change the range of motion that the ultrasound probe 808 to cover a larger or small area of tissue to be imaged, and so on.

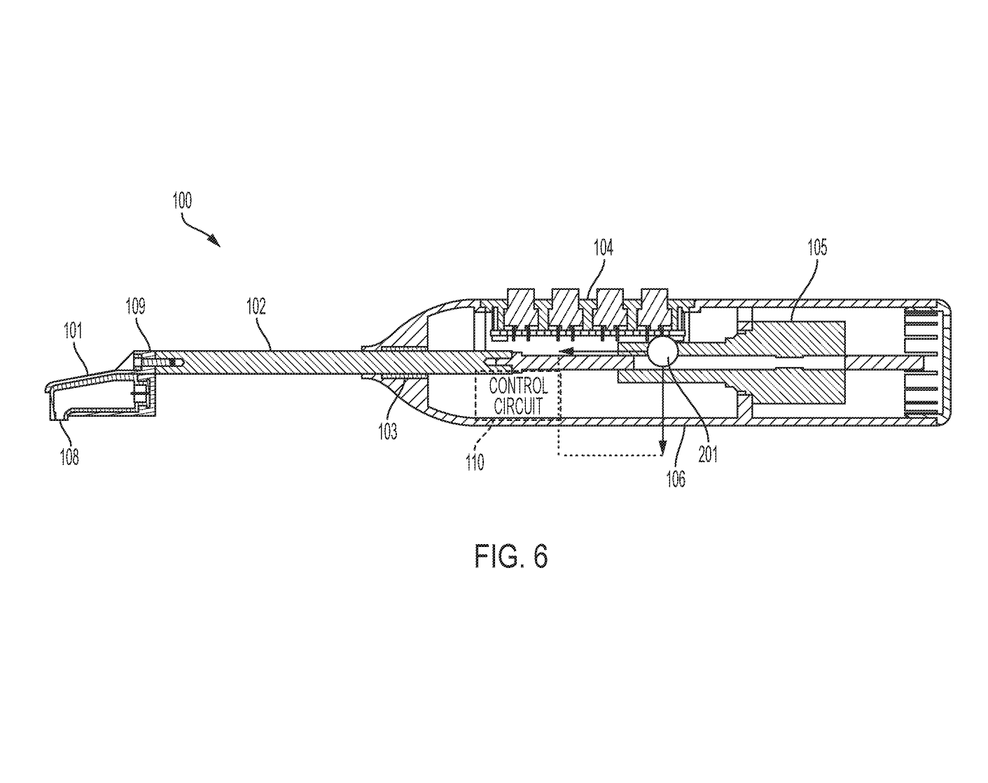

FIG. 6 is a sectional view of the ultrasound oral imaging instrument 100 indicating the center of gravity 201 of the instrument, in accordance with at least one aspect of the present disclosure. In various aspects, the ultrasound oral imaging instrument 100 can be a handheld instrument that has a center of gravity (COG) 201 that makes the ultrasound oral imaging instrument 100 feel comfortable for clinicians to hold and control (i.e., provides improved ergonomics). Accordingly, the ultrasound oral imaging instrument 100 can be constructed such that its COG 201 can be located at approximately the midpoints of the longitudinal and lateral axes of the housing 106. In the depicted aspect, the COG 201 is located roughly below the proximal portion of the user controls 104.

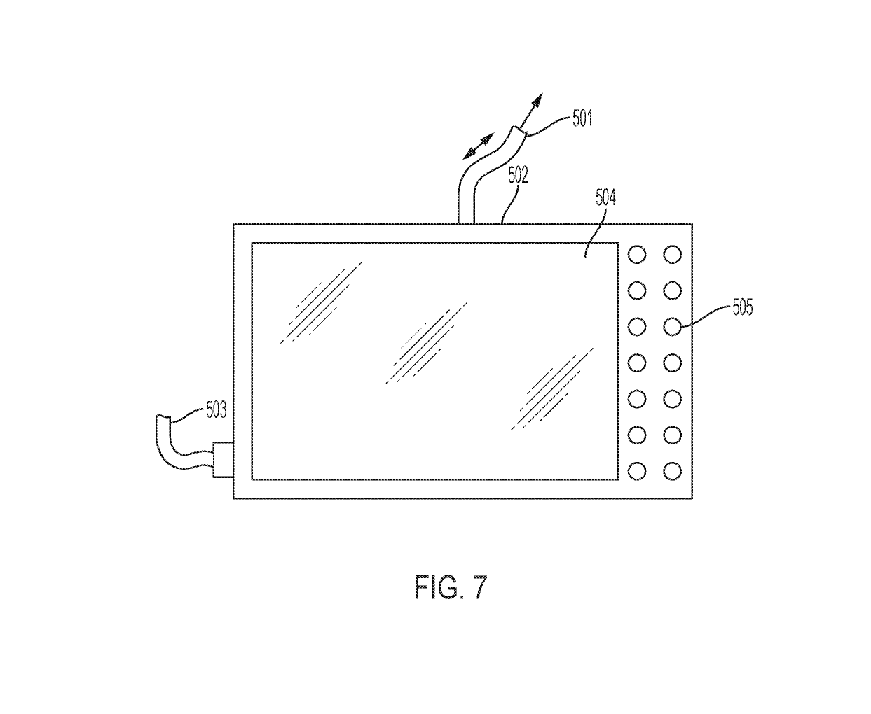

As discussed above, in some aspects the electronic components of the ultrasound oral imaging instrument 100 can be integral to the ultrasound oral imaging instrument 100 (i.e., enclosed within the housing 106). In other aspects, various electronic components can be positioned within a generator or an external control device 502 to which the ultrasound oral imaging instrument 100 is connectable, as depicted in FIGS. 7 and 8, for example. The external control device 502 can include any combination of the control circuit 110 (FIG. 1), motor controller 803 (FIG. 5), power supply 801 (FIG. 5), linear actuator controls 804 (FIG. 5), image reconstruction module 809 (FIG. 5), and any other components described herein. In the depicted aspect, the ultrasound oral imaging instrument 100 is connectable to the external control device 502 via a cable 501. The external control device 502 can further include a touchscreen 504, control panel 505, and/or a power button 605 for activating and controlling the various functions of the ultrasound oral imaging instrument 100 described herein. Power, control signals, and/or data can be transmitted to the ultrasound oral imaging instrument 100 via the cable 501. In other aspects, the external control device 502 can be wirelessly connectable to the ultrasound oral imaging instrument 100 (e.g., via Bluetooth). Further, the external control device 502 can be connectable to a power source via an electrical connector 503, such as a power cord.

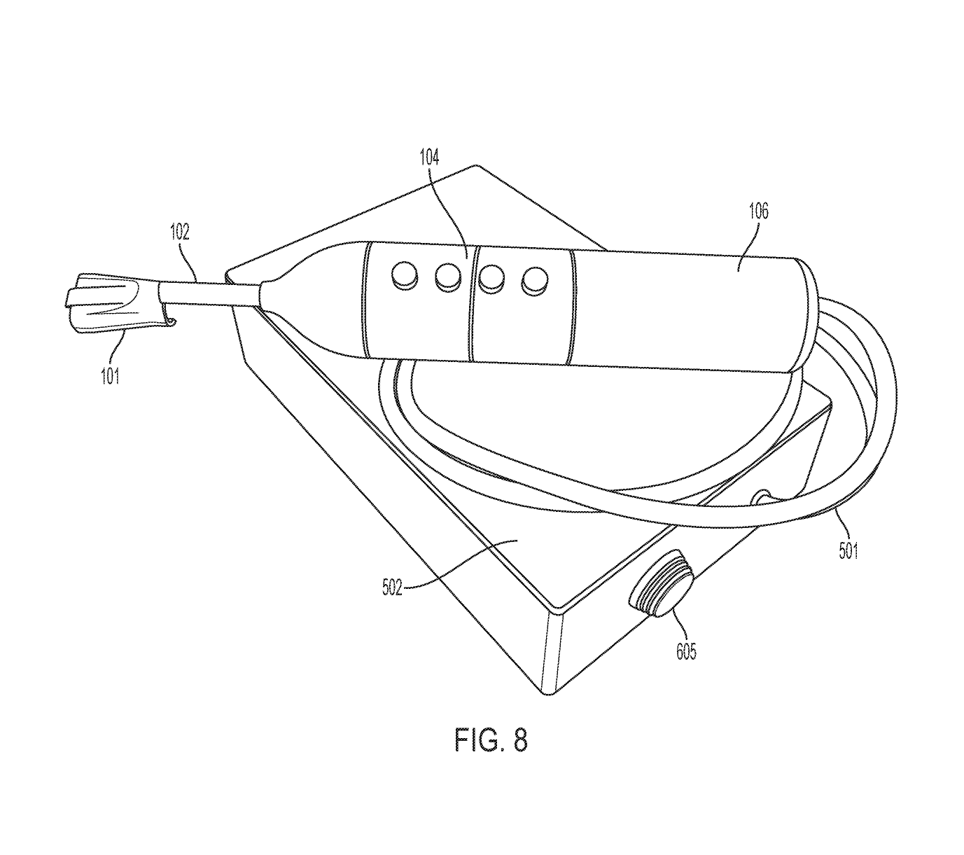

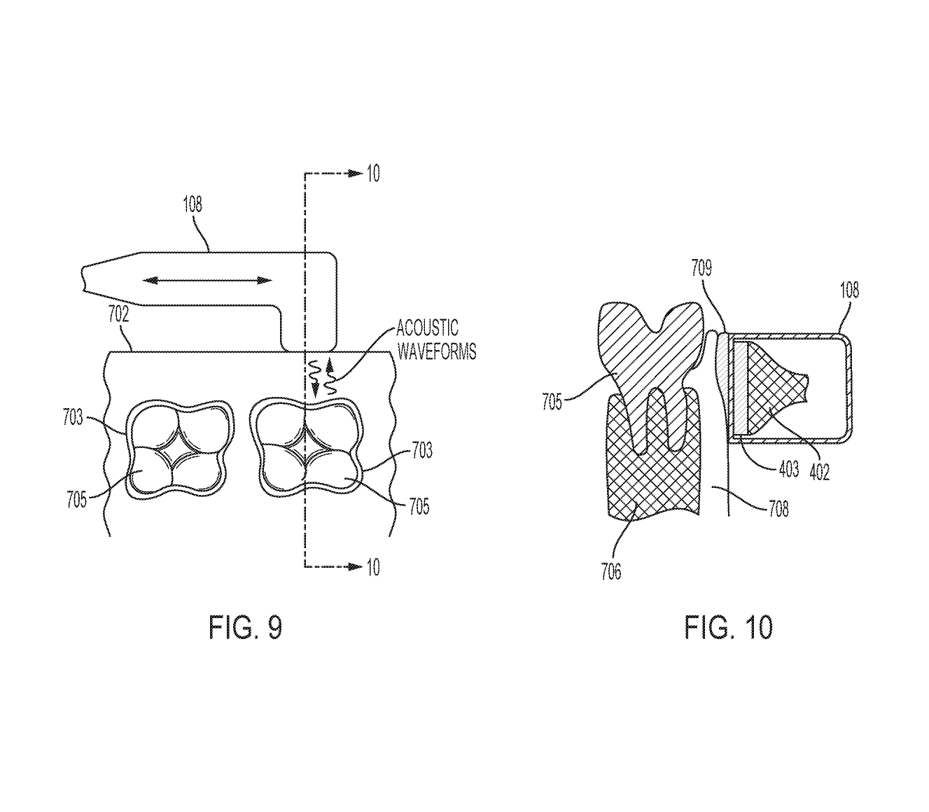

The ultrasound oral imaging instrument 100 described herein has many relevant oral healthcare applications. As noted above, the ultrasound oral imaging instrument 100 can be utilized to image soft oral tissue. In one example described below in connection with FIGS. 9 and 10, the ultrasound oral imaging instrument 100 can be utilized for imaging gingival tissue (i.e., the gingival surface tissue and underlying tissue). In other examples, the ultrasound oral imaging instrument 100 can be utilized for imaging other types of soft oral tissue, such as mucosal tissue, cheek tissue, and/or the tongue. One example application for the ultrasound oral imaging instrument 100 is to image or detect the size of a periodontal pocket. The periodontal pocket is a small space that resides between the teeth and the gingiva. In a healthy person, the periodontal pocket is small. In a person with moderate to severe gingivitis (gum disease), the periodontal pocket is enlarged and inflamed, and the gingiva may recede from the tooth and/or bone. Ultrasound imaging is well-suited for detecting the geometrical characteristics of periodontal pockets and other soft tissue structures.

FIGS. 9 and 10 illustrate a technique for utilizing the ultrasound oral imaging instrument 100 to image soft oral tissue, namely, gingival tissue 708. First, the ultrasound oral imaging instrument 100 is positioned to orient the ultrasound probe 108 against the gingival surface 702 for the gingival tissue 708 to be visualized. The illustrated gingival surface 702 may be a gingival surface anywhere on the internal or external surfaces of the mandibular or maxillary gingiva. Next, the ultrasound oral imaging instrument 100 is activated or otherwise controlled to cause the ultrasound probe 108 to begin simultaneously transmitting ultrasound pulses and moving along the gingival surface 702, as described above. Accordingly, the ultrasonic transducer 402 begins generating ultrasound pulses (e.g., via piezoelectric elements 403 according to a control signal) that are transmitted to the gingival tissue 708, reflected by tissue and/or structures (e.g., bone 706 and teeth 705) within the gingival tissue 708, and then received back by the ultrasound probe 108. In some aspects, an acoustic coupler 709 can be applied to the gingival surface 702 and/or the surface of the ultrasound probe 108 to assist in transmitting the ultrasound pulses between the ultrasound probe 108 and the tissue. For example, the acoustic coupler 709 can include a liquid gel acoustic coupler (e.g., an ultrasound transmission gel) that is spread along the portion of the gingival surface 702 or other tissue that is being imaged. As another example, the acoustic coupler 709 can also include a semi-solid or solid acoustic coupler that is placed and/or fixed directly on or to the ultrasound probe 108 or onto the tissue being imaged.

A 3D reconstruction of the periodontal pocket 703 around a tooth 705 can then be visualized according to 2D slices constructed from ultrasound data indicating the different acoustic characteristics of the bone 706, teeth 705, and/or surrounding gingival tissue 708, as described above. In some aspects, the ultrasound oral imaging instrument 100 can be moved along the gingival surface 702 to visualize an extended area or the ultrasound oral imaging instrument 100 can be held in place or substantially held in place in order to visualize a precise location of the gingival tissue 708. In various aspects, the images captured via the ultrasound probe 108 can be supplied either in real time (i.e., at the time of image acquisition) or at a later time (e.g., stored in a memory of the ultrasound oral imaging instrument 100, an external control device 502, and/or a computer to which the ultrasound oral imaging instrument 100 is communicably connected for playback at a later time).

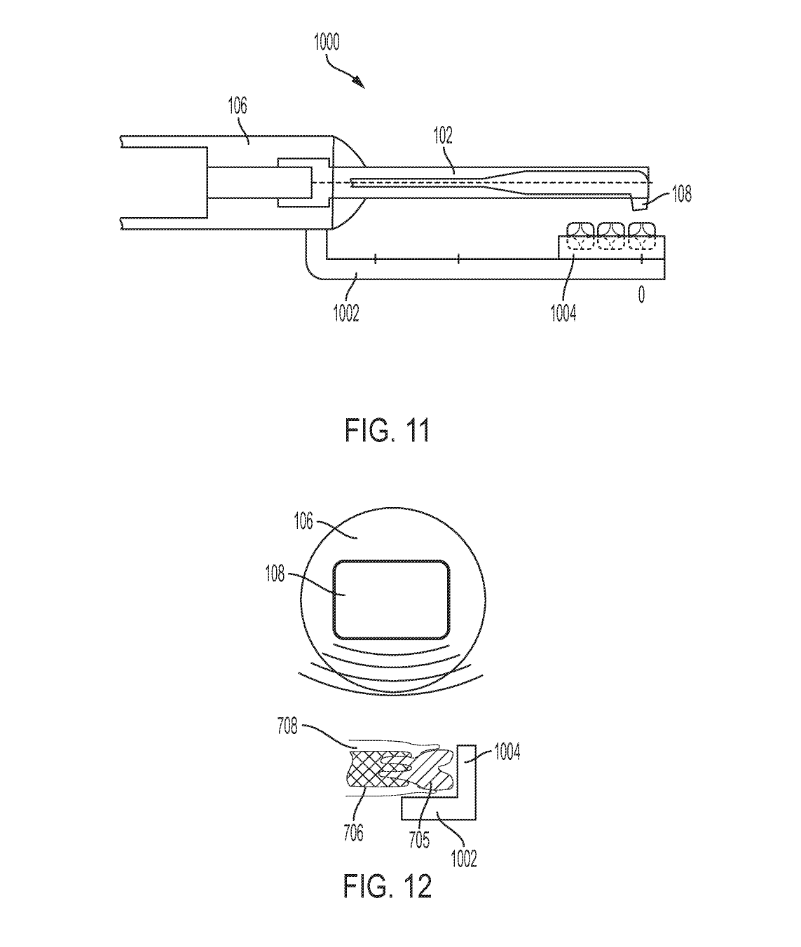

In other aspects, the ultrasound oral imaging instrument 100 can be fixedly held in position relative to the mouth being visualized, rather than being a handheld instrument as indicated in FIGS. 1-10. For example, FIGS. 11 and 12 are views of an ultrasound oral imaging instrument 100 that includes a stabilizer 1002, in accordance with at least one aspect of the present disclosure. In this aspect, the stabilizer 1002 extends from the housing 106 of the ultrasound oral imaging instrument 100 and includes a ledge 1004 against which the operator (e.g., dentist) can stabilize the ultrasound oral imaging instrument 100 relative to the patient. The ledge 1004 can include a chin support against which the operator can stabilize the head of the patient (e.g., as with a panoramic x-ray machine) or, as depicted in FIGS. 11 and 12, a bite piece on which the patient bites to support the ultrasound oral imaging instrument 100 relative to his or her mouth. With the addition of the stabilizer 1002, the depicted aspect of the ultrasound oral imaging instrument 100 can otherwise function as described above in connection with the aspects depicted in FIGS. 1-10.

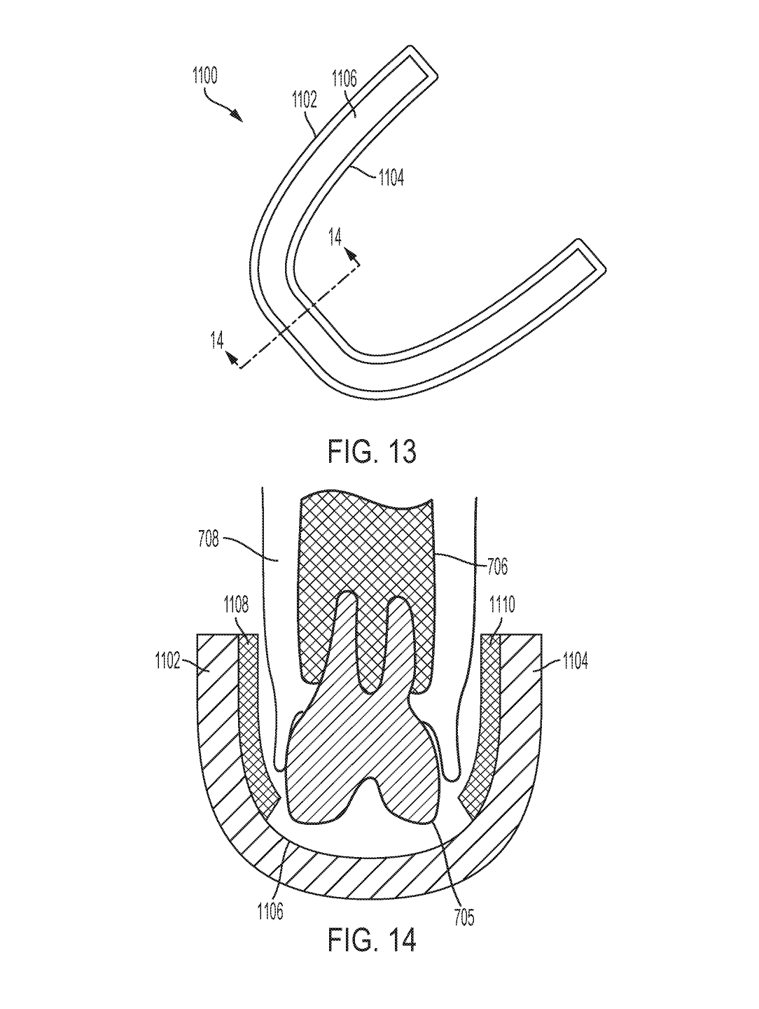

As another example, FIGS. 13 and 14 are views of an ultrasound oral imaging instrument with a general configuration similar to a mouth piece 1100, in accordance with at least one aspect of the present disclosure. In this aspect, the mouth piece 1100 includes a channel 1106 shaped to receive a patient's mouth therein. The mouth piece 1100 further includes one or more ultrasonic transducers 1108, 1110 positioned along the corresponding walls 1102, 1104 of the channel 1106. As described above in the other aspects, the ultrasonic transducers 1108, 1110 can send and receive ultrasound pulses into the tissue enclosed thereby to visualize the gingiva 708, bone 706, and/or teeth 705 therein. As also described above, the transducers 1108, 1110 can include phased arrays of piezoelectric elements in order to control the locations from which the ultrasound pulses are emitted and the orientations of the emitted ultrasound pulses, which can thus be utilized to control the areas within the oral cavity that are being imaged by the instrument. Further, the ultrasonic transducers 1108, 1110 can be coupled to a control circuit that drives the ultrasonic transducer 1108, 1110 and an image reconstruction module for obtaining 3D images of the enclosed tissue. Further, there can be a plurality of ultrasonic transducers 1108, 1110 positioned along each of the opposing walls 1102, 1104 of the mouth piece 1100. As the positions of the ultrasonic transducers 1108, 1110 and/or the piezoelectric elements within the phased arrays are fixed in relation to the mouth piece 1100, the relative positions of the captured 2D slices are known, and 3D images of the tissue can be reconstructed, as described above.

As used in any aspect herein, the term “control circuit” may refer to, for example, hardwired circuitry, programmable circuitry (e.g., a computer processor including one or more individual instruction processing cores, processing unit, processor, microcontroller, microcontroller unit, controller, digital signal processor (DSP), programmable logic device (PLD), programmable logic array (PLA), or field programmable gate array (FPGA)), state machine circuitry, firmware that stores instructions executed by programmable circuitry, and any combination thereof. The control circuit may, collectively or individually, be embodied as circuitry that forms part of a larger system, for example, an integrated circuit (IC), an application-specific integrated circuit (ASIC), a system on-chip (SoC), desktop computers, laptop computers, tablet computers, servers, smart phones, etc. Accordingly, as used herein “control circuit” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory or data storage, e.g., a hard drive), and/or electrical circuitry forming a communications device (e.g., a modem, communications switch, or optical-electrical equipment). Those having skill in the art will recognize that the subject matter described herein may be implemented in an analog or digital fashion or some combination thereof.

As used in any aspect herein, the terms “component,” “system,” “module,” and the like can refer to a computer-related entity, either hardware, a combination of hardware and software, software, or software in execution.

One or more components may be referred to herein as “configured to,” “configurable to,” “operable/operative to,” “adapted/adaptable,” “able to,” “conformable/conformed to,” etc. Those skilled in the art will recognize that “configured to” can generally encompass active-state components, inactive-state components, and/or standby-state components, unless context requires otherwise.

The terms “proximal” and “distal” are used herein with reference to a clinician manipulating the ultrasound oral imaging instrument. The term “proximal” refers to the portion closest to the clinician, and the term “distal” refers to the portion located away from the clinician. It will be further appreciated that, for convenience and clarity, spatial terms such as “vertical,” “horizontal,” “up,” and “down” may be used herein with respect to the drawings. However, the ultrasound oral imaging instruments are used in many orientations and positions, and these terms are not intended to be limiting and/or absolute.

Those skilled in the art will recognize that, in general, terms used herein, and especially in the appended claims (e.g., bodies of the appended claims), are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

It is worthy to note that any reference to “one aspect,” “an aspect,” “an exemplification,” “one exemplification,” and the like means that a particular feature, structure, or characteristic described in connection with the aspect is included in at least one aspect. Thus, appearances of the phrases “in one aspect,” “in an aspect,” “in an exemplification,” and “in one exemplification” in various places throughout the specification are not necessarily all referring to the same aspect. Furthermore, the particular features, structures, or characteristics may be combined in any suitable manner in one or more aspects.

In summary, numerous benefits have been described that result from employing the concepts described herein. The foregoing description of the one or more forms has been presented for purposes of illustration and description. It is not intended to be exhaustive or limiting to the precise form disclosed. Modifications or variations are possible in light of the above teachings. The one or more forms were chosen and described in order to illustrate principles and practical application to thereby enable one of ordinary skill in the art to utilize the various forms and with various modifications as are suited to the particular use contemplated. It is intended that the claims submitted herewith define the overall scope.

1. An ultrasound oral imaging instrument comprising:
an ultrasound probe comprising an ultrasonic transducer;
an actuator coupled to the ultrasound probe, the actuator configured to move the ultrasound probe between a first position and a second position; and
a control circuit coupled to the ultrasonic transducer and the actuator, the control circuit configured to:
cause the ultrasonic transducer to emit ultrasound pulses through the ultrasound probe; and
cause the actuator to move the ultrasound probe between the first position and the second position as the ultrasound probe emits the ultrasound pulses.

2. The ultrasound oral imaging instrument of claim 1, further comprising:
a housing enclosing the actuator and the control circuit; and
a shaft connecting the ultrasound probe to the housing;
wherein the actuator is configured to drive the shaft to move the ultrasound probe between the first position and the second position.

3. The ultrasound oral imaging instrument of claim 2, wherein the ultrasound probe is removably attachable to the shaft.

4. The ultrasound oral imaging instrument of claim 2, further comprising a power source coupled to the ultrasonic transducer and the actuator, wherein the power source is enclosed within the housing.

5. The ultrasound oral imaging instrument of claim 1, wherein the ultrasound oral imaging instrument is connectable to an external power source to power the ultrasonic transducer and the actuator.

6. The ultrasound oral imaging instrument of claim 1, wherein the ultrasound probe is configured to emit the ultrasound pulses perpendicular to an axis along which the ultrasound probe moves between the first position and the second position.

7. The ultrasound oral imaging instrument of claim 1, wherein the ultrasound probe is configured to emit the ultrasound pulses parallel to an axis along which the ultrasound probe moves between the first position and the second position.

8. The ultrasound oral imaging instrument of claim 1, wherein the control circuit causes the ultrasonic transducer to emit ultrasound pulses between at a frequency between 10-65 MHz.

9. A method for obtaining oral images via an ultrasound oral imaging instrument, the method comprising:
applying an acoustic coupler between a gingival tissue of a patient and an ultrasound probe of the ultrasound oral imaging instrument;
moving the ultrasound probe along the gingival tissue, the ultrasound probe comprising a ultrasonic transducer configured to emit an ultrasonic pulse;
capturing a plurality of two-dimensional images via the ultrasound probe as the ultrasound probe is moved along the gingival tissue; and
reconstructing the plurality of two-dimensional images into a three-dimensional image of the gingival tissue.

10. The method of claim 9, wherein the acoustic coupler is applied to the gingival tissue, the acoustic coupler comprising a gel acoustic coupler.

11. The method of claim 9, wherein the acoustic coupler is applied to the ultrasound probe, the acoustic coupler comprising a solid or semi-solid acoustic coupler.

12. The method of claim 9, wherein the gingival tissue comprises gingival surface tissue and underlying tissue.

13. The method of claim 9, wherein the ultrasound probe is moved along the gingival tissue by an actuator coupled to the ultrasound probe.

14. An ultrasound oral imaging instrument comprising:
a mount configured to receive an ultrasound probe, the ultrasound probe comprising an ultrasonic transducer;
an actuator coupled to the ultrasound probe, the actuator configured to move the ultrasound probe between a first position and a second position; and
a control circuit coupled to the ultrasonic transducer, the control circuit configured to:
communicably couple to the ultrasound probe according to whether the ultrasound probe is connected to the mount;
cause the ultrasonic transducer to emit ultrasound pulses through the ultrasound probe; and
cause the actuator to move the mount between the first position and the second position as the ultrasound probe emits the ultrasound pulses.

15. The ultrasound oral imaging instrument of claim 14, wherein the mount comprises a clip.

16. The ultrasound oral imaging instrument of claim 14, further comprising:
a housing enclosing the actuator and the control circuit; and
a shaft connecting the mount to the housing;
wherein the actuator is configured to drive the shaft to move the mount between the first position and the second position.

17. The ultrasound oral imaging instrument of claim 14, wherein the ultrasound oral imaging instrument is connectable to an external power source to power the ultrasonic transducer and the actuator.

18. The ultrasound oral imaging instrument of claim 14, wherein the mount is oriented such that the ultrasound probe emits the ultrasound pulses perpendicular to an axis along which the mount oscillates between the first position and the second position.

19. The ultrasound oral imaging instrument of claim 14, wherein the mount is oriented such that the ultrasound probe emits the ultrasound pulses parallel to an axis along which the mount oscillates between the first position and the second position.

20. The ultrasound oral imaging instrument of claim 14, wherein the control circuit causes the ultrasonic transducer to emit ultrasound pulses between at a frequency between 10-65 MHz.
